(19) United States
(12) Patent Application Publication
(10) Pub. No.: US 2025/0035634 A1
(43) Pub. Date: Jan. 30, 2025

(54) A METHOD FOR DETECTING CLUSTERING OF RAS PROTEIN

(71) Applicants: Nanyang Technological University, Singapore (SG); University Medical Center of the Johannes Gutenberg University Mainz, Mainz (DE)

(72) Inventors: Wenting ZHAO, Singapore (SG); Huanwen MU, Singapore (SG); Krishnaraj JALINGAM, Mainz (DE)

(73) Assignees: Nanyang Technological University, Singapore (SG); University Medical Center of the Johannes Gutenberg University Mainz, Mainz (DE)

(21) Appl. No.: 18/717,849

(22) PCT Filed: Dec. 9, 2022
(86) PCT No.: PCT/SG2022/050896

§ 371 Date: Jun. 7, 2024

(30) Foreign Application Priority Data
Dec. 9, 2021 (SG) 10202113656R

(51) Int. Cl. G01N 33/574; G01N 33/50; G01N 33/58

(52) U.S. Cl. CPC G01N 33/5748; G01N 33/5008; G01N 33/582; G01N 2333/914

(57) ABSTRACT

The present invention relates to a method of detecting clustering of Rat Sarcoma Vims (Ras) protein in a cell comprising culturing the cells on an array of nanostructures and detecting the clustering of Ras protein around the nanostructures by using optical microscopy. In one embodiment, the nanostructures are nanobars. The invention further relates to the use of the method in identifying the isoform or mutation status of Ras protein, and anti-Ras drug screening.

### TECHNICAL FIELD

The present invention relates generally to methods of biomolecular detection. Disclosed are methods for detecting protein clustering using optical microscopy, and uses of the methods thereof in drug screening.

### BACKGROUND

Ras oncogenes are known contributors to dysregulation in cell proliferation, differentiation and survival, and are implicated in around 20% of human cancers. It remains challenging to develop effective anti-Ras drugs for clinical use despite efforts over the past decades. The traditional strategy of directly inhibiting the enzymatic activity of Ras has been unfruitful due to the high affinity of Ras for guanosine triphosphate (GTP) (in the picomolar range), and the lack of deep pockets in the enzymatic G-domain of Ras for small molecule docking. Alternative inhibitory strategies targeting the binding between Ras and its effectors and the activity of downstream signaling proteins have been complicated by the intertwined Ras signaling pathways and sometimes defeated by compensatory mechanisms. One promising anti-Ras strategy focuses on the interaction between Ras and the plasma membrane, which controls the initiation of Ras signaling. Ras has been reported to segregate into GTP-dependent dimers and higher-order clusters on the plasma membrane, which are critical for their effector activation, and inhibiting Ras dimerization was demonstrated effectively abolished Ras signaling in one previous study. As such, tools to evaluate inhibitors of Ras clustering are under demand to facilitate anti-Ras drug screening.

It would be desirable to overcome or alleviate at least one of the above-described problems, or at least to provide a useful alternative.

### SUMMARY

Disclosed herein is a method of detecting clustering of a Rat Sarcoma Virus (Ras) protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference indicates the isoform or mutation status of Ras.

Disclosed herein is a method of identifying the isoform or mutation status of Ras in a cell, the method comprising: a) culturing the cell on an array comprising a plurality of nanostructures; and b) visualizing the Ras protein using optical microscopy to detect clustering of the Ras protein around the plurality of nanostructures, wherein the Ras protein is labelled, and wherein a change in the degree of Ras clustering as compared to a reference indicates the isoform or mutation status of Ras.

Disclosed herein is a method of predicting the biological activity of Ras protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference predicts the biological activity of Ras in the cell.

Disclosed herein is a method of identifying a subject suffering or is likely to be suffering from a cancer associated with a Ras mutation and/or isoform, the method comprising: a) culturing a cell from the subject on an array comprising a plurality of nanostructures; and b) visualizing the Ras protein using optical microscopy to detect clustering of the Ras protein around the plurality of nanostructures, wherein the Ras protein is labelled, and wherein a change in the degree of Ras clustering as compared to a reference identifies the subject as one suffering or is likely to be suffering from a cancer associated with a Ras mutation and/or isoform.

Disclosed herein is a method of screening for a drug compound, the method comprising: a) culturing a cell on an array comprising a plurality of nanostructures; b) contacting the cell with a candidate drug compound, c) visualizing Ras protein using optical microscopy to detect an increase or decrease in the degree of clustering of the Ras protein around the plurality of nanostructures as compared to a reference, wherein the Ras protein is labelled.

Disclosed herein is a method of predicting the efficacy of an anti-Ras drug on a subject, the method comprising: a) culturing a cell that is obtained from the subject on an array comprising a plurality of nanostructures; b) contacting the cell with the anti-Ras drug, c) visualizing Ras protein using optical microscopy to detect an increase or decrease in the degree of clustering of the Ras protein around the plurality of nanostructures as compared to a reference, wherein the Ras protein is labelled, wherein an increase or decrease in the degree of clustering of the Ras protein predicts the efficacy of the anti-Ras drug on the subject.

### BRIEF DESCRIPTION OF THE DRAWINGS

Embodiments of the present invention will now be described, by way of non-limiting example, with reference to the drawings in which:

FIG. 1 shows a workflow for nanostructure-based Ras cluster detection: 1) fabrication of the nanostructured-chip (here displaying nanobars as an example); scale bar, 3 μm for nanobar array, 1 μm for single nanobar images by scanning electron microscopy (SEM). 2) cell culture on nanobars visualized by Cell Mask plasma membrane staining dye; 3) Ras labelling; 4) anti-Ras inhibitor treatment; 5) fluorescence imaging and image analysis for pattern comparison. Scale bar, 10 μm for cell overview, 3 μm for inset of the fluorescent images displayed.

FIG. 2 shows the accumulation of K-Ras clusters on nanobars in a live cell. (a) U2OS cells expressing EGFP-KRasWT with (bottom left) and without (top left) EGF activation (100 ng/ml, 1 hr). Arrowheads highlight the K-Ras clusters accumulated at the nanobar ends. The corresponding averaged images and surface plots of the cells displayed are shown on the right (88 nanobars for without activation, 25 nanobars for with activation). Scale bar, 3 μm. (b) Scatter plot of Enrichment Index (EI) of the KRasWT intensity at nanobar ends over the flat sidewall with and without EGF activation. Data is shown as mean±SD with all data points displayed. Welch's t test (2 tailed, unpaired, assuming unequal SD) was performed for statistical analysis. **** represents p<0.0001. n=1165 for without activation and n=1251 for with activation.

FIG. 3 shows nanobar-guided clustering of oncogenic KRasG12V, KRasG13D and KRasQ61K compared to KRasWT in cells. (a) Fluorescent images of U2OS cells expressing EGFP-tagged KRasWT (top left), KRasG12V (top right), KRasG13D (bottom left) and KRasQ61K (bottom right) on nanobars. Scale bar, 10 μm for cell overview, 3 μm for inset. The corresponding averaged surface plots are shown at the corner (245 nanobars for KRasWT, 1386 nanobars for KRasG13D, 1059 nanobars for KRasG12V and 126 nanobars for KRasQ61K). (b) Violin plot of the EI of KRasWT, KRasG13D, KRasG12V and KRasQ61K. Data is shown as median with upper/lower quartile. Kolmogorov-Smirnov tests (unpaired, nonparametric) were performed for statistical analysis. * represents p<0.05, ** for p<0.01, and **** for p<0.0001. Mean±s.e.m. equal to 1.78+0.063, 1.90+0.031, 1.87+0.035 and 1.96+0.072 with n=437, 2564, 2124 and 328 for KRasWT, KRasG13D, KRasG12V and KRasQ61K respectively. (c) Relative frequency distributions of EI of KRasWT, KRasG13D, KRasG12V and KRasQ61K with Lognormal fitting curves.

FIG. 4 shows the colocalization between K-Ras variants and active Ras probe CRaf-RBD on nanobars. (a) Fluorescent images of U2OS cells cotransfected with K-Ras variants and RFP-CRaf-RBD. Normalized intensity profiles of the highlighted regions in the inset images are shown at right (line width: 4 pixels). Scale bar, 10 μm for cell overview, and 3 μm for inset. Arrowheads point the colocalized signals on nanobars between two channels. (b) Scatter plot of the Pearson correlation coefficient between K-Ras variants and CRaf-RBD respectively (n=93, 101 and 36 nanobars for KRasS17N, KRasWT and KRasG12V, shown in mean±SD). An unpaired t test (assuming equal SD) was used. **** represents p<0.0001.

FIG. 5 shows nanobar-guided clustering of three Ras isoforms with the same oncogenic G12V mutation. (a) Fluorescent images of U2OS cells expressing EGFP-tagged KRasG12V (left), NRasG12V (middle) and HRasG12V (right) on nanobars. The corresponding averaged images and surface plots of the cells displayed are shown below (43 nanobars for KRasG12V, 18 nanobars for NRasG12V and 22 nanobars for HRasG12V). Scale bar, 10 μm for cell overview, 3 μm for inset. (b) Box plot of Enrichment Index (EI) of KRasG12V, NRasG12V and HRasG12V. Data is shown as median with upper/lower quartile, and +marks mean. The upper and lower limits are of 90% CI. Welch's t test (2 tailed, unpaired, assuming unequal SD) was performed for statistical analysis. ** represents p<0.01 and for p<0.0001. n=1691 for KRasG12V, n=888 for NRasG12V and n=2657 for HRasG12V. (c) Relative frequency distribution of EI of KRasG12V, NRasG12V and HRasG12V with Gaussian fitting curves. The differences of each pair are plotted separately below with the difference in EI distribution highlighted in light yellow.

FIG. 6 shows the effect of Ras inhibitor rocaglamide on nanobar-guided K-Ras and H-Ras clustering. (a) Fluorescent images of U2OS cells expressing KRasG12V (top) and HRasG12V (bottom) with rocaglamide (right) and DMSO treatment (control, left) on nanobars. The corresponding averaged images and surface plots of the cells displayed are shown below (80 and 69, 28 and 58 nanobars for DMSO and rocaglamide treatment of KRasG12V and HRasG12V respectively). Scale bar, 10 μm for cell overview, 3 μm for inset. (b) Box plot of Enrichment Index (EI) of HRasG12V and KRasG12V with the treatment of DMSO (2 μL/2 mL, 4 hrs for HRasG12V and 1 hr for KRasG12V) and rocaglamide (100 nM, 4 hrs for HRasG12V and 1 hr for KRasG12V). Mean±s.e.m. equal to 1.93±0.029 and 1.87±0.029 for DMSO and rocaglamide treatment of HRasG12V; 1.73±0.028 and 1.58±0.027 for DMSO and rocaglamide treatment of KRasG12V. n=1789, 2223, 1412 and 1073 respectively. Data is shown as median with upper/lower quartile, and ±marks mean. The upper and lower limits are of 90% CI. Welch's t test (2 tailed, unpaired, assuming unequal SD) was performed for statistical analysis. **** represents p<0.0001 and ns for no significant difference. Detection time (c) and detection limit (d) of rocaglamide on KRasG12V clustering in line plots. Data is shown as mean±s.e.m., equal to 1.94±0.053, 1.72±0.032, 1.55±0.021 and 1.64±0.024 for DMSO (1 μL/2 mL, 4 hrs) and rocaglamide treatment (50 nM under 15 mins, 1 hr and 4 hrs); 1.73±0.028, 1.64±0.018, 1.55±0.021 and 1.58±0.027 for DMSO (2 μL/2 mL, 1 hr) and rocaglamide treatment (25 nM, 50 nM and 100 nM under 1 hr). n=735, 1306, 2496 and 1369 for (c); n=1412, 2704, 2496 and 1073 for (d) respectively. The corresponding averaged images and surface plots of the batches of nanobars (577, 709, 1298 and 525 nanobars for (c); 724, 1436, 1298 and 843 nanobars for (d) from left to right) are shown below. Statistical significance of differences between control group of DMSO treatment and rocaglamide-treated cells were measured using Welch's tests as well, with ** for p<0.01, *** for p<0.001 and **** for p<0.0001.

FIG. 7 shows IC50 data of rocaglamide on nanobar-guided oncogenic K-Ras clustering. A dot plot of the EI of KRasG12V with a gradient concentration of rocaglamide. Data is shown as mean±s.e.m., equal to 1.73±0.028, 1.64±0.018, 1.55±0.021 and 1.58±0.027 for DMSO (2 μL/2 mL, 1 hr) and rocaglamide treatment (25 nM, 50 nM and 100 nM, 1 hr). n=1412, 2704, 2496 and 1073 respectively. One-phase decay fitted curve was displayed for IC50 calculation using Prism9.

FIG. 8 shows example workflows using the present invention. (a) Workflow using nanobar chips applied for high-throughput anti-Ras drug screening; (b) Workflow using nanobar chips applied for biopsy-enabled cancer treatment.

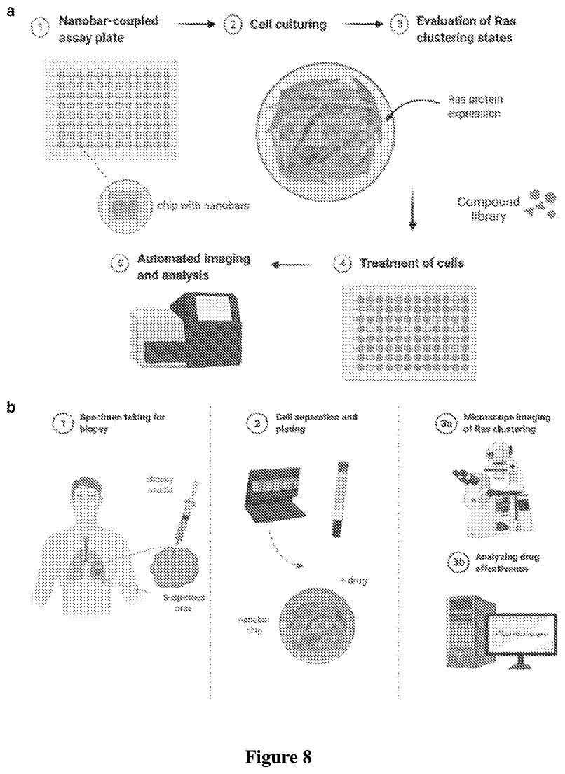

FIG. 9 shows the accumulation of K-Ras and H-Ras clusters on gradient nanobars in live cells. (a) SEM images in top view of gradient nanobars ranging from 200 nm to 1050 nm with 600 nm in height. Scale bar, 2 μm. Fluorescent images of U2OS cells expressing EGFP-KRasG12V (b) and HRasG12V (d) on the gradient nanobars in the view of brightfield (left) and fluorescence (right). Scale bar, 10 μm. The averaged signals of KRasG12V and tK-Ras (c) and of HRasG12V and tH-Ras (d) over multiple nanobars of the diameters ranging from 200 nm to 1050 nm with a step size of 50 nm. Scale bar, 2 μm. The intensity profiles (line width: 7 pixels) of each and their overlays are displayed below for visualization.

### DETAILED DESCRIPTION

The present invention teaches a method of detecting clustering of a labelled protein in a cell, the method comprising: a) culturing the cell on an array comprising a plurality of nanostructures; and b) visualizing the labelled protein using optical microscopy to detect clustering of the labelled protein around the plurality of nanostructures. In some embodiments, the labelled protein is a membrane-associated protein.

Disclosed herein is a method of detecting clustering of a Rat Sarcoma Virus (Ras) protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference indicates the isoform or mutation status of Ras.

Without being bound by theory, certain cellular proteins may preferentially cluster around sites with nanoscale curvatures. The present disclosure relates to the use of nanostructure arrays with defined geometry and curved shape to guide protein clusters into quantifiable patterns along the contours of the nanostructures. This may enable the differentiation of the clustering of different proteins and protein variants using optical microscopy instead of other microscopy techniques like electron microscopy (EM) and FLIM-FRET (fluorescence lifetime imaging microscopy-fluorescence resonance energy transfer).

As used herein a protein “cluster” refers to a local concentration of proteins, and protein “clustering” refers to the formation of a local concentration of proteins. A protein cluster may form due to protein-protein interactions, which may be covalent (e.g., through the formation of disulphide bonds) or non-covalent (e.g., through electrostatic interactions, hydrogen bonding, hydrophobic interactions, etc.). Intrinsic features of a protein may drive clustering, including but not limited to spatial features, e.g., hydrophobic patches; conformational features, e.g., binding pockets, secondary structures like helices or beta-sheets, conformational changes induced by an upstream molecular signal; sequence features, e.g., cysteine residues, a series of charged residues; protein modifications, e.g. phosphorylation, lipidation, proteolytic cleavage, etc.; or a combination thereof. Alternatively, protein clustering may be driven by local cellular conditions, e.g., by spatial constraints within the cell or within the plasma membrane, or by a specific configuration of one or more cellular components or compartments to which the protein is bound. Clustering may also result from intermolecular interactions of a second protein to which a protein is bound. A group of proteins in a cluster may be identical or may comprise one or more modified forms of a protein, e.g., a mutant protein, a post-translationally modified protein, a protein isoform, a protein splice variant, etc. A protein cluster may also comprise different proteins each with a similar or a different function. A protein cluster may take on a specific aggregate spatial conformation or be a loose collection of proteins. As used herein, a multimer is a kind of protein cluster consisting of two or more subunits of the same protein or of a group of protein isoforms. In the context of this disclosure, proteins in a cluster retain their native conformation and any native function they may possess.

A “membrane-associated protein” of this disclosure refers to any protein having a domain that stably attaches to or associates with the plasma membrane (i.e. a membrane-association domain), where such domains may include myristoylated domains, palmitoylated domains, prenylated domains, transmembrane domains, lipid-anchoring domains, lipid-binding domains and the like. Specific membrane-associated proteins of interest include, but are not limited to: myristoylated proteins, e.g., flotillins; farnesylated proteins, e.g., Ras, Rheb; proteins binding specific lipid bilayer components like phosphatidylserine, phosphatidylinositol or cholesterol, e.g., HER2, CEA; transmembrane proteins, e.g., EpCAM, EGFR. Membrane-associated proteins include, but are not limited to, proteins that contain large regions or structural domains that are embedded in or bound to the membrane, and also include proteins that only have a small region bound to the membrane wherein the bulk of the protein is on the cytoplasmic and/or exoplasmic side of the membrane. Exemplary membrane-associated proteins include but are not limited to proteins that are permanently attached to the plasma membrane (e.g., integral membrane proteins), and proteins that adhere only temporarily to the plasma membrane (e.g., peripheral membrane proteins). Membrane-associated proteins often form complexes that mediate essential cellular processes e.g., signal transduction, transport and cell-cell communication. Accordingly, a membrane-associated protein of this disclosure may be bound to one or more proteins, and all of these proteins may be different. Membrane-associated proteins may also assemble into higher order oligomers and nanoclusters while associated with the plasma membrane. This assembly may be mediated by the membrane-associated domain, or by one or more cytoplasmic and/or exoplasmic domains. In some instances assembly is necessary for the effective functioning of the membrane-associated protein, e.g., it may improve downstream signaling efficiency. In one embodiment, the membrane-associated protein is a Ras protein.

As used herein “Ras” or “Rat Sarcoma Virus protein” refers to a protein in the Ras superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, and Miro subfamily. The most notable members of the Ras subfamily are H-Ras, K-Ras and N-Ras, mainly for being implicated in many types of cancer. However, there are many other members including DIRAS1; DIRAS2; DIRAS3; E-Ras; GEM; M-Ras; NKIRAS1; NKIRAS2; N-Ras; RALA; RALB; RAPIA; RAPIB; RAP2A; RAP2B; RAP2C; RASD1; RASD2; RASL10A; RASL10B; RASL11A; RASL11B; RASL12; REM1; REM2; RERG; RERGL; RRAD; R-Ras; and R-Ras2. As used herein a “Ras isoform” refers to H-Ras, K-Ras or N-Ras, and splice variants thereof. H-Ras is also referred to as HRas, HRAS or H-RAS herein. N-Ras is also referred to as NRas, NRAS or N-RAS herein. K-Ras is also referred to as KRas, KRAS or K-RAS herein.

“Mutation status” of Ras refers to the one or more amino acid mutations which may be present in a Ras isoform. The mutation status may be established with respect to a common reference sequence such as a wild-type (WT) amino acid sequence. A mutation may be an introduced mutation, a naturally occurring mutation, or a non-naturally occurring mutation. In some embodiments, a mutation may be a substitution (e.g., a substituted amino acid), insertion (e.g., addition of one or more amino acids), or deletion (e.g., removal of one or more amino acids). Two or more mutations may be consecutive, non-consecutive, or a combination thereof. The mutation may be a gain-of-function or a loss-of-function mutation. In some embodiments, a mutation may be present at any position of Ras. A mutant Ras may be a full-length or truncated polypeptide. A Ras mutant may be a dominant negative mutant. In some embodiments, the mutation status of Ras is a point substitution in codon 12, 13 or 61 (or a combination thereof) of K-Ras, H-Ras or N-Ras.

In some embodiments the Ras protein is an endogenous or a heterologous Ras protein. As used herein, the term “endogenous” means that the protein is expressed from a gene naturally found in the genome of the cell, and the term “heterologous” means that the protein is not expressed from a gene naturally found in the genome of the cell. A heterologous protein may be expressed from a gene vector introduced exogenously to the cell, e.g., a plasmid vector, adeno-associated viral vector, lentiviral vector, etc. The membrane-associated protein may be a mutant of a wild-type protein, such as a deletion mutant or a point mutant, and may show a gain of function or loss of function. The mutation in a mutant membrane-associated protein may have occurred naturally or may be one that has been engineered, e.g., through genetic engineering of the gene for the wild-type protein. The membrane-associated domain may also be a mutant of a wild-type domain, e.g., a wild-type domain that has been engineered for improved membrane binding, or a wild-type domain that has been truncated. A membrane-associated protein may be a chimera of a membrane-associated domain and one or more other protein domains, e.g., the protein may be a chimera containing a detection domain like a fluorescent protein or an enzymatic reporter.

In some embodiments the protein is labelled with a fluorescence tag. As used herein, the terms “fluorescence tag”, “fluorescence label” and “fluorescence probe” are used interchangeably to refer to a fluorescent entity that is bound to a protein to aid in the optical detection of said protein. Examples of such fluorescent entities include but are not limited to fluorescent proteins (e.g., green fluorescent protein, allophycocyanin, and wavelength-shifted variants thereof), non-protein organic fluorophores (e.g., fluorescein and other xanthene derivatives) and semiconducting nanocrystals. The fluorescent tag may selectively bind to a specific region or functional group on the protein (e.g., a tetracysteine motif, a hexahistidine motif, an epitope, etc.) or it may be attached chemically or biologically. Chemical attachment may be via a reaction with a free sulfhydryl or a free amine moiety of the protein, or via another reactive chemical moiety (e.g., carboxyl, azide) that has been conjugated to the protein. Biological attachment may be via genetically-engineered protein fusion.

In certain embodiments the fluorescence tag is a fluorescent protein that is genetically fused to the protein or membrane-associated protein under study in the methods herein. In other embodiments the fluorescence tag is fused to an antibody that recognises the protein under study, or fused to a secondary antibody that recognises a primary antibody bound to the protein under study. In some embodiments, the cell is fixed and/or permeabilized to introduce the protein label. Cells may be fixed with chemical crosslinkers or cold alcohol, and permeabilized with detergents. Fixation and permeabilization should minimise disruption to protein clusters that are present at the point of fixation and/or permeabilization. Permeabilization conditions may be optimized to minimise disruption. Permeabilization conditions to be optimized include the nature of the detergent used, the detergent concentration, length of detergent treatment, etc. Alternatively, the step of permeabilization may be omitted.

As used herein a “nanostructure” or “nanopillar” is an erect structure which is vertically aligned with respect to the substrate it is attached to. A nanostructure may be vertically aligned at an angle normal to the plane of the substrate (90°), or at angles substantially close to 90°. The plurality of nanostructures may be identical.

In some embodiments the plurality of nanostructures comprises a combination of curved and flat surfaces. As used herein a curved surface is a surface that produces a curved section when the nanostructure is viewed in an optical or electron microscopy image, and a flat surface is a surface that produces a straight section when the nanostructure is viewed in an optical or electron microscopy image. A curved surface may produce one or more curved sections when viewed in an optical or electron microscopy image, and a flat surface may produce one or more straight sections when viewed in an optical or electron microscopy image. In some embodiments each nanostructure comprises a combination of curved and flat surfaces. Each nanostructure may comprise one or more curved surface and/or one or more flat surface. A curved surface on a nanostructure may be adjacent to a flat surface. In preferable embodiments a nanostructure with a curved surface is adjacent to a nanostructure with a flat surface. A nanostructure may contain one or more branch points or arms. Each branch point or arm may comprise a combination of curved and flat surfaces.

The length of each nanostructure is at least the resolution of the optical microscope used. The length and width of each nanostructure does not exceed the length of a cell to be cultured in the method herein. The resolution of an optical microscope is defined as the shortest distance between two points on a specimen that can still be distinguished as separate in an optical microscopy image. An optical microscope may, for example, have a resolution of about 300 nm in the x-y plane. The typical length of a mammalian cell is between 10 microns and 20 microns. In one embodiment the length of a nanostructure is 2 microns.

The nanostructures may each have a height of at least 0.3 microns. The nanostructures may have a height between about 0.3 microns to about 20 microns. The nanostructures may have a height of about 0.3 microns, about 0.4 microns, about 0.5 microns, about 0.6 microns, about 0.7 microns, about 0.8 microns, about 0.9 microns, about 1.0 microns, about 1.1 microns, about 1.2 microns, about 1.3 microns, about 1.4 microns, about 1.5 microns, about 1.6 microns, about 1.7 microns, about 1.8 microns, about 1.9 microns, about 2.0 microns, about 2.5 microns, about 3 microns, about 3.5 microns, about 4 microns, about 4.5 microns, about 5 microns, about 5.5 microns, about 6 microns, about 6.5 microns, about 7 microns, about 7.5 microns, about 8.0 microns, about 8.5 microns, about 9.0 microns, about 9.5 microns, about 10 microns, about 10.5 microns, about 11 microns, about 11.5 microns, about 12 microns, about 12.5 microns, about 13 microns, about 13.5 microns, about 14 microns, about 14.5 microns, about 15 microns, about 15.5 microns, about 16 microns, about 16.5 microns, about 17 microns, about 17.5 microns, about 18 microns, about 18.5 microns, about 19 microns, about 19.5 microns or about 20 microns. In one embodiment, the nanostructures have a height of about 0.3 microns.

In some embodiments a curved surface has an arc width of less than about 1050 nm. The arc width is a general term in the art referring to the length of a straight line between the two ends of an arc, or the length of a straight line between two points of a curvature. A curved surface may have an arc width of about 10 nm to about 1050 nm, about 20 nm to about 1000 nm, about 50 nm to about 800 nm, about 100 nm to about 600 nm, 150 nm to about 400 nm, about 200 nm to about 300 nm. A curved surface may have an arc width of about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, or about 1050 nm. In some embodiments a curved surface has an arc width of about 200 nm to about 900 nm, about 200 nm to about 950 nm, about 200 nm to about 1000 nm, or about 200 nm to about 1050 nm. In one embodiment a curved surface has an arc width of about 250 nm.

The plurality of nanostructures may be positioned at regular intervals from one another. The nanostructures may have a pitch that is more than 0.2 microns. The nanostructures may have a pitch of between about 0.2 microns and about 8 microns, between about 0.5 microns and about 6 microns, between about 1 microns and about 5 microns, between about 2 microns and about 4 microns, or about 3 microns. In one embodiment, the nanostructures have a pitch of between about 2.0 microns, about 2.1 microns, about 2.2 microns, about 2.3 microns, about 2.4 microns, about 2.5 microns, about 2.6 microns, about 2.7 microns, about 2.8 microns, about 2.9 microns, about 3.0 microns, about 3.1 microns, about 3.2 microns, about 3.3 microns, about 3.4 microns, about 3.5 microns, about 3.6 microns, about 3.7 microns, about 3.8 microns, about 3.9 microns, about 4.0 microns, about 4.1 microns, about 4.2 microns, about 4.3 microns, about 4.4 microns, about 4.5 microns, about 4.6 microns, about 4.7 microns, about 4.8 microns, about 4.9 microns or about 5.0 microns. In one embodiment, the nanostructures have a pitch of about 5.0 microns.

The plurality of nanostructures may be etched in regular arrays on a substrate using known etching methods, e.g., electron beam lithography (EBL), reactive ion etching, etc. The nanostructure and the substrate it is attached to may be constructed of any material that is appropriate for a particular method of etching. The nanostructure and substrate may be constructed of the same or different materials. The nanostructures are preferably monolithic, such as formed from a single substrate material. In some embodiments the nanostructures and substrate are quartz. In some embodiments the nanostructures are etched using electron beam lithography. In some embodiments all the nanostructures are identical.

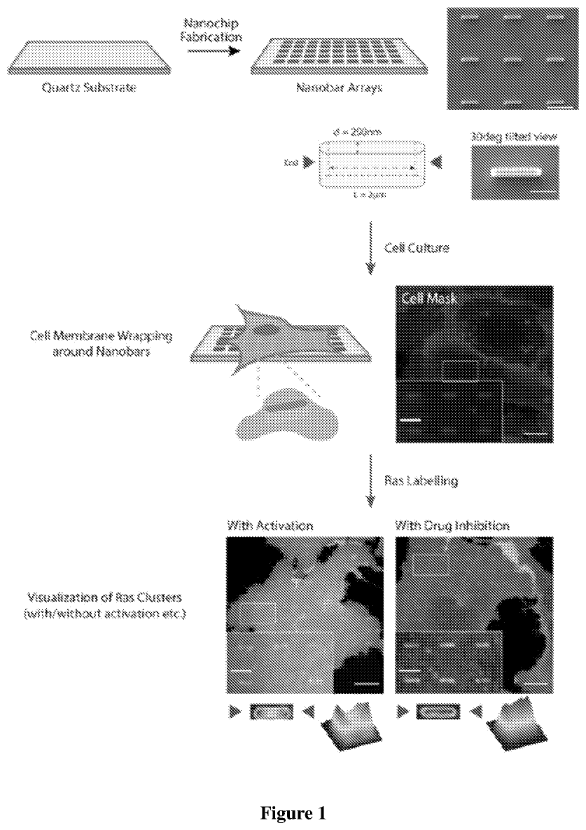

In some embodiments the nanostructures are nanobars. As used herein a nanobar is a nanostructure wherein the longest dimension is parallel to the substrate surface. The arc width of a nanobar may also be the width of the nanobar. In some embodiments the bar has a pill shape when viewed under optical or electron microscopy, as seen in FIG. 1.

With the method of this disclosure, each nanostructure may provide at least one data point, wherein each data point may relate to the difference in protein clustering at a curved section compared to protein clustering at a straight section of the nanostructure. An optical image for analysis may cover around a hundred μm² surface area and may include tens to over a hundred data points per cell, allowing both individual protein clusters and the clustering state across the whole cell surface to be evaluated.

A data point may be a quantitative parameter that defines the degree of protein clustering at a curved section relative to a straight section of a nanostructure. In some embodiments this parameter is an Enrichment Index. As used herein, an “Enrichment Index” or “EI” refers to the degree of protein clustering at a curved section compared to the degree of clustering at a straight section of a nanostructure. Each nanostructure may provide multiple EIs depending on the number of curvatures and linear sections it contains. In some embodiments a numerical average or the frequency distribution of the EIs provided by all the nanostructures covered by a cell is used as a quantitative readout of the degree of clustering for a particular Ras protein. The EI values of different Ras proteins may be compared to differentiate the Ras proteins. Each Ras protein may have an EI signature, where such a signature will depend on the implementation of the method herein (e.g., the dimension and/or arrangement of the nanostructures, the Ras label used, the optical microscopy technique employed, etc.). The skilled practitioner will appreciate that the evaluation of more EIs increases the accuracy and sensitivity of protein cluster characterization. Moreover, the number of EIs collected for each cell may be adjusted by varying the parameters of the nanostructure array, like the pitch size.

In one embodiment, a change in the degree of Ras clustering as compared to a reference indicates the isoform or mutation status of Ras. For example, the Ras protein may be wild-type or have a G12V, G13D or Q61K mutation. The Ras protein may be a K-Ras, N-Ras or H-Ras isoform.

In one embodiment, a change in the degree of Ras clustering as compared to a reference indicates the mutation status of Ras. The change in the degree of Ras clustering may indicate whether Ras has wild-type activity (i.e. 100% biological activity) or mutant activity (i.e. less than or greater than 100% biological activity. A mutant activity may indicate the presence of a Ras mutation such as a G12V, G13D or Q61K mutation.

The reference referred to herein may be the degree of clustering of a wild-type Ras protein (for distinguishing mutants), or a specific Ras isoform (for distinguishing isoforms). The reference may also be a predetermined value or an average value. The change in Ras clustering may be an increase or a decrease in the degree of clustering around the nanostructures, as visualized on microscopy, as compared to the reference. In some embodiments a change in the degree of clustering is represented by a change in the calculated Enrichment Index (EI) as compared to a reference. Without being bound by theory, a larger EI value for a Ras mutant or isoform may indicate a greater degree of clustering around the nanostructures.

In one embodiment, the method distinguishes between wild-type WT, G12V, G13D or Q61K Ras. The method may distinguish wild-type WT from oncogenic mutants such as G12V, G13D or Q61K Ras. Alternatively, the method may distinguish between different oncogenic mutants G12V, G13D or Q61K Ras.

In one embodiment, the method distinguishes between the oncogenic G12V mutants of K-Ras, N-Ras or H-Ras.

As used herein, the term “increase” or “increased” with reference to the degree of clustering (e.g., as calculated by the EI) may refer to a statistically significant and measurable increase in the degree of clustering as compared to a reference. The increase may, for example, be an increase of at least about 10%, or an increase of at least about 20%, or an increase of at least about 30%, or an increase of at least about 40%, or an increase of at least about 50%, or more.

As used herein, the term “decrease” or “decreased” with reference to the degree of clustering (e.g., as calculated by the EI) may refer to a statistically significant and measurable decrease as compared to a reference. The decrease may, for example, be a decrease of at least about 10%, or a decrease of at least about 20%, or a decrease of at least about 30%, or a decrease of at least about 40%, or a decrease of at least about 50%.

Disclosed herein is method of identifying the isoform or mutation status of Ras in a cell, the method comprising: a) culturing the cell on an array comprising a plurality of nanostructures; and b) visualizing the Ras protein using optical microscopy to detect clustering of the Ras protein around the plurality of nanostructures, wherein the Ras protein is labelled, wherein a change in the degree of Ras clustering as compared to a reference indicates the isoform or mutation status of Ras.

Disclosed herein is a method of predicting the biological activity of Ras protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference predicts the biological activity of Ras in the cell. The method may, for example, predict that the Ras has lower or higher activity than wild-type Ras. Hence, the method may predict whether the cell is likely to contain a wild-type or a mutant Ras. The biological activity of a Ras protein may be, for example, its ability to form a complex with one or more downstream effector proteins (e.g., Raf), or its ability to activate one or more downstream effector pathways.

In one embodiment, the method provides an indication whether the subject is suffering from cancer or is likely to be suffering from cancer due to a particular Ras mutation or isoform. For example, the method may identify that the subject is suffering from a cancer due to a KRasG12V, KRasG13D or KRasQ61K mutation. The method may identify that the subject is suffering from a cancer due to a NRasG12V, NRasG13D or NRasQ61K mutation. The method may identify that the subject is suffering from a cancer due to a HRasG12V, HRasG13D or HRasQ61K mutation.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a sub-stage of a Stage II cancer. One skilled in the art will appreciate that the classification of a Stage II cancer as either an early stage cancer or a late stage cancer depends on the particular type of cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof.

In one embodiment, the cancer cell is a solid or haematological cancer cell.

The term “haematological cancer′ may refer to one or more of leukemia, lymphoma, Chronic Myeloproliferative Disorders, Langerhans Cell Histiocytosis, Multiple Myeloma/Plasma Cell Neoplasm, Myelodysplasia Syndromes, Myelodysplastic/Myeloproliferative Neoplasms or a combination thereof. In some embodiments, leukemia is any one or more of Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia (HCL) or a combination thereof. In some embodiments, lymphoma is any one or more of AIDS-Related Lymphoma, Cutaneous T-Cell Lymphoma, Hodgkin Lymphoma, Mycosis Fungoides, Non-Hodgkin Lymphoma, Primary Central Nervous System Lymphoma, Sezary Syndrome, T-Cell Lymphoma, Cutaneous, Waldenstrom Macroglobulinemia or a combination thereof.

The term “solid cancer” may refer to one or more of breast cancer, large intestinal cancer, lung cancer, small cell lung cancer, gastric (stomach) cancer, liver cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, esophageal cancer, small intestine cancer, endocrine cancer, thyroid cancer, parathyroid cancer, adrenal cancer, soft tissue tumor, urethral cancer, penile cancer, prostate cancer, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer, sarcomas or a combination thereof.

Disclosed herein is a method of identifying a subject suffering or is likely to be suffering from a cancer associated with a Ras mutation and/or isoform, the method comprising: a) culturing a cell from the subject on an array comprising a plurality of nanostructures; and b) visualizing the Ras protein using optical microscopy to detect clustering of the Ras protein around the plurality of nanostructures, wherein the Ras protein is labelled, wherein a change in the degree of Ras clustering as compared to a reference identifies the subject as one suffering or is likely to be suffering from a cancer associated with a Ras mutation and/or isoform.

In some embodiments the cell is treated with a drug molecule or candidate drug molecule. The drug or candidate drug may be a biological, pharmaceutical, or chemical compound. Non-limiting examples include a simple or complex organic or inorganic molecule, a peptide, a protein, an oligonucleotide, an antibody, an antibody derivative, antibody fragment, a vitamin derivative, a carbohydrate, a toxin, and a chemotherapeutic compound. Various compounds can be synthesized, for example, small molecules and oligomers (e.g., oligopeptides and oligonucleotides), and synthetic organic compounds based on various core structures. In addition, various natural sources can provide drug compounds, such as plant or animal extracts, and the like. The drug or candidate drug may be administered on its own or as an active ingredient in a pharmaceutical composition. As used herein a drug or candidate drug also refers to combinations of two or more drugs or candidate drugs. The cell may be treated with the drug before it is cultured on the nanostructure array, or the drug may be added during cell culture on the nanostructure array. Where the drug is added during cell culture, the method offers a real-time readout of the drug effects in individual cells.

The drug molecule or candidate drug molecule may affect protein clustering. For instance, it may enhance or prevent cluster formation, disperse existing clusters, change the size of clusters, change the rate of cluster formation, change the membrane location of clusters, change the pattern of clustering locally or throughout the cell, among other effects. The skilled person will recognize that certain treatment parameters may require optimization for each drug or drug combination, where these parameters include but are not limited to the drug concentration, length of drug exposure, and frequency of drug application.

In some embodiments a change in the degree of Ras clustering as compared to a reference provides an indication of the specificity and/or sensitivity of an anti-Ras drug molecule or candidate drug molecule.

The reference referred to herein may be a cell that has not been treated with a drug or candidate drug, or a cell that has been treated with a different drug or candidate drug, or treated with a different combination of drugs, or treated with a different drug regimen, e.g., a different drug concentration, different length of treatment, etc.

As used herein an “anti-Ras drug” or an “anti-Ras candidate drug” refers to a drug molecule or a candidate drug molecule which, when administered alone or as an active ingredient in a pharmaceutical composition, inhibits one or more biological functions of a Ras isoform or mutant. Non-limiting examples of biological functions include Ras expression, localization, activation, and interaction with other proteins. While preferred drugs or candidate drugs herein specifically interact with a target Ras, compounds that inhibit a biological activity of the target Ras by interacting with other members of a signal transduction pathway of which the target Ras is a member are also specifically included within this definition. Effects of anti-Ras drugs or candidate drugs include but are not limited to: silencing Ras gene expression; preventing Ras protein synthesis; inhibiting Ras trafficking; inhibiting Ras membrane localization; preventing Ras activation (e.g., by trapping the protein in an inactive conformation, or preventing nucleotide exchange, etc.); inhibiting interaction of Ras with a downstream effector protein; preventing the formation of higher-order Ras complexes; targeting Ras for intracellular degradation; or a combination thereof. In one embodiment the anti-Ras drug is rocaglamide.

As used herein “specificity” or “selectivity” of an anti-Ras drug or candidate drug refers to the ability of the drug or candidate drug to target a specific Ras isoform or mutant. Thus a drug is specific to or selective for a Ras isoform or mutant if it only targets that Ras isoform or mutant, and is non-specific or non-selective otherwise. The “IC50” referred to herein is the drug concentration which achieves a half-maximal inhibition of protein clustering in a cell. Drug “sensitivity” as used herein relates to the method of drug testing and is defined to be the lowest drug concentration or treatment time that produces a quantifiable effect on protein clustering in the cell. The skilled person will recognize that drug sensitivity as defined herein depends on the nature of the drug-protein interaction and on the parameters of the method of drug testing, and is not a property inherent to the drug itself. For the method of this disclosure, parameters that may affect drug sensitivity include but are not limited to: the label used for the protein, the method of labelling the protein; the cell type used for testing; and the number of nanostructures available for analysis for each cell.

Also disclosed herein is a method of screening for a drug compound, the method comprising: a) culturing a cell that contains a labelled protein on an array comprising a plurality of nanostructures; b) contacting the cell with a candidate drug compound, c) visualizing the labelled protein using optical microscopy to detect a difference in the degree of clustering of the labelled protein around the plurality of nanostructures as compared to a reference. In some embodiments the protein under study is a membrane-associated protein.

The reference referred to herein may be a cell that has not been treated with the drug, or a cell that has been treated with a different drug, or treated with a different combination of drugs, or treated with a different drug regimen, e.g., a different drug concentration, different length of treatment, etc.

In some embodiments the nanostructure array is fabricated on quartz microchips, and the chips are placed on or integrated into individual compartments of an assay plate to increase the throughput of drug screening. In some embodiments the imaging and protein clustering analysis is automated.

In some embodiments the drug compound is one that is capable of promoting or inhibiting clustering of the labelled protein.

In some embodiments the cell is one that has been obtained from a subject. The subject may be a mammal such as a human, or a non-human mammal. In some embodiments the subject is a patient, and the cell is obtained from a biopsy of the patient.

In some embodiments the drug screen is used to identify an effective drug (or combination of drugs) for treating a subject. As used herein an “effective drug” or “effective combination of drugs” refers a drug or drug combination that achieves an intended level of inhibition of protein clustering when used in the methods of this disclosure. Accordingly, a drug (or drug combination) is effective if it achieves an intended level of inhibition of protein clustering, and less effective if it achieves some level of inhibition of protein clustering, and ineffective if it does not change the clustering of a protein. Drug effectiveness may depend on the subject and the disease condition being treated, e.g., the weight and age of the subject, the severity of the disease condition, the manner of administration and the like, which can readily be determined by one of ordinary skill in the art. Drug effectiveness will also depend on the dosing regimen to be followed, whether it is administered in combination with other compounds, timing of administration, the cell to which it is administered, and the physical delivery system in which it is carried.

Disclosed herein is a method of predicting the efficacy of an anti-Ras drug on a subject, the method comprising: a) culturing a cell that is obtained from the subject on an array comprising a plurality of nanostructures; b) contacting the cell with the anti-Ras drug, c) visualizing Ras protein using optical microscopy to detect an increase or decrease in the degree of clustering of the Ras protein around the plurality of nanostructures as compared to a reference, wherein the Ras protein is labelled, wherein an increase or decrease in the degree of clustering of the Ras protein predicts the efficacy of the anti-Ras drug on the subject.

The method may further comprise treating the subject suffering from cancer.

The term “treating” as used herein may refer to (1) delaying the appearance of one or more symptoms of the condition; (2) inhibiting the development of the condition or one or more symptoms of the condition; (3) relieving the condition, i.e. causing regression of the condition or at least one or more symptoms of the condition; and/or (4) causing a decrease in the severity of the condition or of one or more symptoms of the condition.

It will be appreciated that many further modifications and permutations of various aspects of the described embodiments are possible. Accordingly, the described aspects are intended to embrace all such alterations, modifications, and variations that fall within the spirit and scope of the appended claims.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Methods

### Design, Fabrication and Preparation of Nanochips

Arrays of nanostructures on SiO₂ surface of bar shape are fabricated with two curved ends and flat sidewalls using electron beam lithography (EBL). For demonstration below, each nanobar is of 2 μm long, 300 nm height and the two curved ends are two half circles with a diameter of 250 nm (FIG. 1, top). To conduct EBL, the chips are spin-coated with an electron-sensitive layer, 300 nm of positive electron-beam resist poly (methyl methacrylate) (PMMA) (MicroChem), followed by a conductive polymer layer of AR-PC 5090.02 (Allresist). Desired patterns are then written on the polymer through exposure to EBL (FEI Helios NanoLab), and developed in isopropanol:methylisobutylketone (IPA:MIBK)=3:1 solution. Subsequently, a 30-nm chromium (Cr) mask is deposited onto the surface via thermal evaporation (UNIVEX 250 Benchtop) and lift-off in acetone. Nanostructures are generated through reactive ion etching with the mixture of CF4 and CHF3 (Oxford Plasmalab 80). After fabrication, scanning electron microscopy (SEM) (FEI Helios NanoLab) imaging can be performed after 10-nm Cr coating to measure the dimensional properties of nanostructures (FIG. 1, top). Before use, the nanostructured-chip is cleaned by Chromium Etchant (Sigma-Aldrich) to remove the remaining metal layer.

### Cell Culture on Nanobar Chips

To culture the cells onto the nanostructured chip, the chip is first cleaned by air plasma treatment (Harrick Plasma) and sterilized by UV radiation. Additional coating reagents like extracellular matrix (ECM) protein components (e.g., fibronectin or gelatin, Sigma-Aldrich) can be added to promote cell attachment. After coating, the cells are plated and maintained in DMEM (Gibco) supplemented with fetal bovine serum (FBS, Life Technologies) and Penicillin-Streptomycin (Life Technologies) in a standard incubator at 37° C. with 5% CO2.

### Fluorescence Tagging of Ras Proteins Route 1—Transient Expression of Ras Proteins in Cells

The Ras proteins can be expressed transiently by transfecting the fluorescent protein-tagged Ras plasmid (e.g., EGFP-KRasG12V, EGFP, enhanced green fluorescent protein) into cells using the Lipofectamine (Life Technologies) protocol or electroporation (e.g., Neon Transfection System, Thermo Fisher). If necessary, the transfection parameters (e.g., the amount of the plasmid, starvation time and expression time) and electroporation parameters (e.g., pulse voltage, pulse width, pulse number) can be optimized to reach the expected transfection efficiency and expression level. FBS can be removed at the expression step from the culture medium to prevent Ras activation.

### Fluorescence Tagging of Ras Proteins Route 2—Immunostaining of Endogenous Ras Proteins in Cells

Immunostaining labels endogenous Ras proteins and thus is useful for personalized treatment. After cell fixation, the permeabilization of the plasma membrane is optional or to be optimized to minimize the disruption of the Ras proteins localized on the plasma membrane. Then the Ras proteins are recognized with the antibody specific to Ras (Invitrogen) and the secondary antibody with fluorescence tag enabling microscopic fluorescence imaging. If any drug treatment is to be conducted, the immunostaining should be done after the treatment.

### Treatment of Anti-Ras Inhibitor on Cells

To evaluate the effect of a Ras inhibitor on disrupting the Ras clusters, a gradient concentration and several treatment durations can be selected to determine the effective window. A control group with no treatment should be conducted at the same time. Before and after treatment of the same batch of cells can also be compared.

### Fluorescence Imaging and Quantification of Ras Clusters Around Nanobars

Cell imaging is performed with laser scanning confocal microscopy (Zeiss LSM 800 with Airyscan) and a Plan-Apochromat 100×/1.4 oil objective. EGFP is excited at 488 nm and detected at 490-570 nm. RFP is excited at 561 nm and detected at 570-645 nm. Images were taken with a resolution of 512×512 pixels, 16-bit and 124 nm pixel size. A scanning speed of 20 s/image and an averaging mode of 4 times/line can be applied. During imaging, the cells are maintained in live cell imaging solution (Thermo Fisher) at 37° C. in an on-stage incubator. To demonstrate the nanobar-guided Ras patterning using epifluorescence microscopy, the modular DMi8 inverted microscope (Leica Microsystems), equipped with ORCA-Flash4.0 LT scientific CMOS camera (Hamamatsu Photonics, Japan) and Leica ×100 oil immersion objective lens (NA 1.4) can be used. The images are conducted with a single snap shot for display of Ras variants and the quantification. For the imaging of co-expressed K-Ras variants and CRaf-RBD, z-stack images can be taken and averaged for display and the intensity profiles. The calculation of Pearson correlation coefficient is also based on single snap shot.

To quantify the nanobar-guided clustering of Ras variants, the fluorescence intensity ratio at the nanobar end to the flat sidewall (i.e. Enrichment Index, EI) is calculated. The background signals of each image are firstly subtracted by the rolling ball algorithm in ImageJ (radius=3 pixels for 250 nm diameter nanobars). Next, the nanobars covered by the cell to be analyzed are identified by a custom-written MATLAB (MathWorks) code. The positions of each nanobar with the two ends and the center can be anchored at the same time and are verified with ImageJ. Thus, the signal intensities at the nanobar ends and the center can be extracted automatically for EI calculation, resulting in two data points per nanobar. Nanobars with undetectable protein signals or with the signals blocked by random aggregates are excluded. To minimize the possible human bias, all the parameters for the calculation and the sorting criteria for data validation have been kept consistent throughout the entire quantification process. After the data validation, the remaining data points are ready for plotting. The box plot and the frequency distribution curves of the EI values can be made by Prism9.

To calculate the Pearson correlation coefficient, the background signals in the fluorescent images beside the nanobar regions ares firstly removed by rolling ball at a 9-pixel radius in Image J. Then the coefficient was obtained through the plugin Coloc 2 with each data point generated from an individual nanobar.

Five examples are described below to demonstrate how the nanobar arrays can differentiate the clustering change of different Ras isoforms and oncogenic mutants using conventional fluorescence microscopes (laser scanning confocal microscope, Zeiss LSM800, is used for demonstration).

### Example 1

### Detection of Wild-Type K-Ras Clustering Change Upon Activation by Epidermal Growth Factor (EGF)

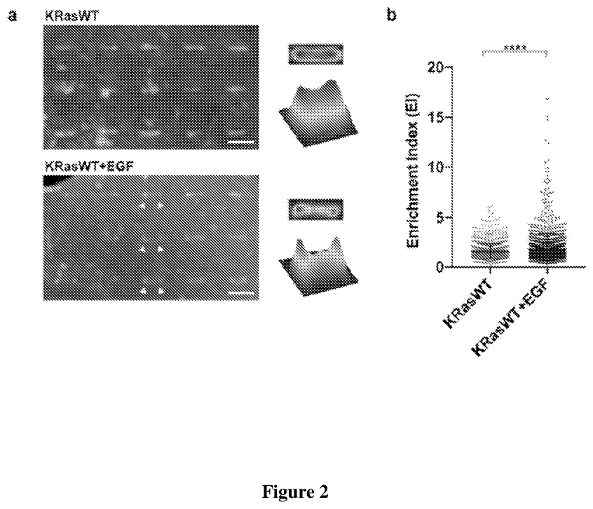

To visualize the Ras clusters around nanobars, U2OS cells are cultured on fibronectin-coated nanobar arrays (2 μg/ml, 30 min, 37° C.) and transfected with EGFP-tagged K-Ras4B wild-type (KRasWT) for overnight expression. Since Ras are known to change their clustering status upon activation for downstream signaling, the cells are activated with EGF treatment of 100 ng/ml for 1 hr. As shown in FIG. 2a, the active K-Ras clusters under EGF condition are found specifically accumulated to the ends of nanobars, while those without EGF treatment do not show obvious spatial segregation around nanobars. Therefore, the nanobar design is effective in generating visible patterns of K-Ras clusters upon EGF treatment, overcoming the challenge to achieve nanoscale imaging resolution for Ras cluster characterization.

To quantify the clustering state difference at the nanobar ends, the bar-end to bar-center signal intensity ratio of Ras, i.e. Enrichment Index, EI, is calculated over around 600 nanobars using a customized Matlab code to provide a quantifiable readout for evaluation. As shown in FIG. 2b, KRasWT without EGF treatment (1.59+0.023, hereafter all in mean±s.e.m.,) can be significantly differentiated from those without EGF treatment (1.89+0.042, p<0.0001).

### Example 2

### Detection of Clustering Changes Among Oncogenic Mutations of K-Ras

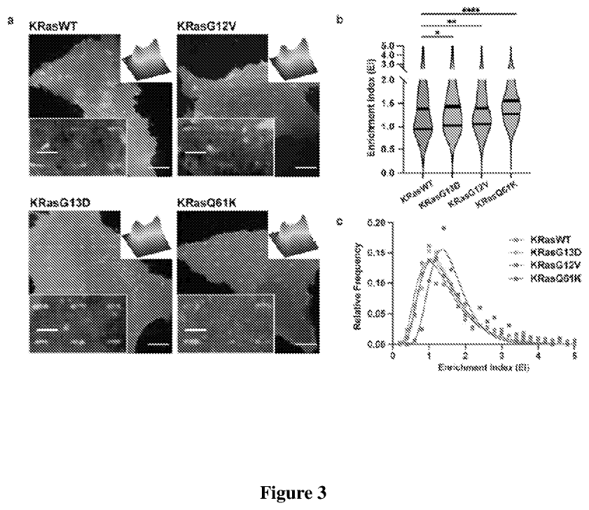

K-Ras is known to host more than ten oncogenic mutations and carries the highest mutation rate in human cancer. With each mutation providing divergent contributions to different types of cancers, quantitative differentiation of their behaviours offers great advantages in developing mutant-specific therapeutics. The capability of nanobars is therefore validated in characterizing the clusters of different Ras mutants. Specifically, three oncogenic mutants of K-Ras, KRasG12V, KRasG13D and KRasQ61K tagged with EGFP, are transfected into U2OS cells separately for comparison with KRasWT. As shown in FIG. 3a, the oncogenic mutants tested are found displaying more preferential cluster accumulation at nanobar ends compared to wild-type, based on the average intensity plots displayed over hundreds of nanobars. The quantified EIs further reveal their preference to accumulate at the nanobar ends in the order of KRasQ61K (1.96+0.072)>KRasG13D (1.90+0.031)>KRasG12V (1.87+0.035)>KRasWT (1.78+0.063) (FIG. 3b). In addition, their responses to nanobar guidance can also be compared by plotting the frequency distribution curves of their EIs (FIG. 3c), the right shift of which indicates a higher percentage of larger cluster formation at nanobar ends. As shown, right shifts of the distribution curves of KRasG13D, KRasG12V and KRasQ61K can be observed compared to that of KRasWT. Especially, KRasQ61K shifts most to the right than KRasG13D and KRasG12V. Therefore, the oncogenic KRasQ61K shows the highest curvature-guided clustering followed by KRasG13D and KRasG12V. Hence, this result proves that the nanobar-based analysis can manifest the subtle difference between oncogenic mutants and provide quantitative readout to differentiate their behaviours on the plasma membrane.

### Example 3

Detection of Ras Activation States by the Colocalization Test with the Active Ras-Labelling Probe

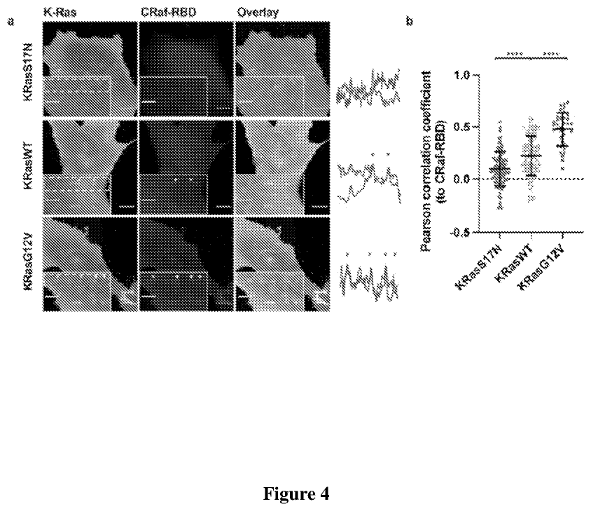

Ras activation states can also be probed by a well-established active Ras sensor, the Ras binding domain (RBD) of Ras immediate effector C-Raf (CRaf-RBD). Thus, the activation states of Ras can be inferred by testing the colocalization between Ras and CRaf-RBD on nanobars. To demonstrate, EGFP-K-Ras variants of different activation states are coexpressed with red fluorescent protein (RFP)-tagged CRaf-RBD in cells plated on nanobar arrays. Specifically, the constitutively active KRasG12V, and the dominant negative KRasS17N are compared with KRasWT. As shown in FIG. 4, in cells expressing the dominant negative mutant KRasS17N, although KRasS17N shows detectable signals due to surface area increase via membrane wrapping, CRaf-RBD displays no detectable signals, confirming no active Ras on nanobars. In contrast, when expressing KRasWT, CRaf-RBD exhibits much higher signals on the nanobars, indicating the presence of active Ras in the nanobar-guided Ras clusters. An interesting observation here is that the KRasG12V signals exhibits good colocalization with CRaf-RBD signals across the entire nanobar (FIG. 4a, bottom row), while KRasWT only shows partial correlation with differential intensity distributions across the nanobar region (FIG. 4a, middle row). KRasS17N, as expected, exhibits no correlation (FIG. 4a, top row). Quantitative characterization of the presence of active Ras on the nanobars can be achieved by calculating the Pearson correlation coefficient between the images of the two channels as displayed in FIG. 4b. In result, the colocalization between Ras and CRaf-RBD on nanobars is found to be the highest for KRasG12V (mean±SD, 0.48±0.16) followed by the reduced colocalization for KRasWT (0.22±0.19) and no colocalization for KRasS17N close to 0 (0.10±0.16). These observations are in accordance with activation states of the three Ras variants, confirming the ability of nanobar-generated Ras patterns to quantitatively differentiate Ras in different activation states.

### Example 4

### Detection of the Clusters of Different Ras Isoforms, H-Ras and N-Ras

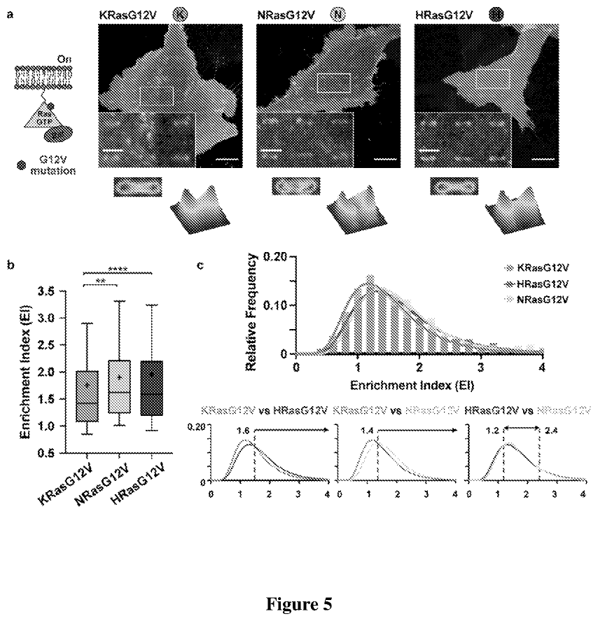

On top of K-Ras, Ras proteins have two other isoforms, i.e. H-Ras and N-Ras, the mutations of which are also discovered in different cancer types. The similar oncogenic mutants of different isoforms, i.e. HRasG12V and NRasG12V are also compared with KRasG12V. Each of them is tagged with EGFP and transiently expressed into U2OS cells on nanobars. After characterization, all three isoforms similarly show enhanced fluorescence signals at the nanobar ends (FIG. 5a), indicating their preferential accumulation to curved membrane sites. In addition, the EIs quantified from over 400 to 1300 nanobars reveal the differential clustering ability among them. As shown in FIG. 5b, HRasG12V and NRasG12V generate significant higher EI values than KRasG12V (HRasG12V, 1.96±0.028; NRasG12V, 1.90±0.040; KRasG12V, 1.75±0.029), suggesting the H- and N-Ras isoforms are more sensitive to membrane curvatures. It is consistent with previous EM-based studies using H-Ras proteins and truncated c-terminals of H-Ras on nanobars. More intriguingly, the frequency distribution of their EIs further reveal the subtle difference among H-Ras and N-Ras that has not been reported. As shown in FIG. 5c, although the distribution curves of both HRasG12V and NRasG12V shift to the right of KRasG12V, their shifts are derived from different regions of the EI. HRasG12V generates higher EI than KRasG12V starting from EI at 1.6; while NRasG12V starts a bit earlier from 1.4. It suggests that HRasG12V and NRasG12V prefer to form bigger clusters than KRasG12V at the nanobar ends and those bigger clusters formed by NRasG12V are slightly more than HRasG12V. Further comparison between HRasG12V and NRasG12V curves shows that the discrepancy of these two isoforms is mainly located from 1.2<EI<2.4 with NRasG12V on the top of HRasG12V, confirming the higher tendency of NRasG12V in forming bigger clusters on curved membrane sites. Overall, the results demonstrate that nanobar arrays-guided Ras patterning can sensitively differentiate the three Ras isoforms by testing their clustering behaviours on nanobar-curved membranes in cells.

### Example 5

### Evaluation of Isoform Specificity and Sensitivity of Anti-Ras Drug

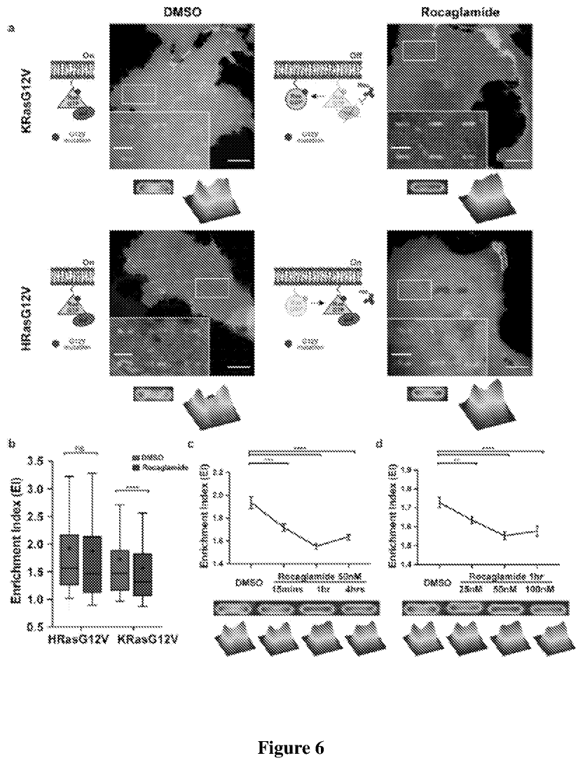

The ability to evaluate drug specificity toward different isoforms and the sensitivity at different drug concentrations are critical for effective drug screening. Here, these aspects of the invention are demonstrated using a small molecular K-Ras inhibitor, rocaglamide. Interestingly, comparing the two isoforms with same mutations KRasG12V and HRasG12V (FIG. 6a), significant reduction of bar-end accumulated-clustering for KRasG12V was observed upon drug treatment, resulting in a uniform distribution of Ras signal across the nanobar similar to the non-activated KRasWT without EGF shown earlier (FIG. 2a). But, for HRasG12V, no obvious changes can be observed with the same drug treatment, which strongly indicates that this invention can be used to evaluate the drug specificity among different Ras species. Statistical analysis of their EI values on nanobars further confirms the inhibition specificity of rocaglamide on K-Ras over H-Ras measured by the nanobar platform (FIG. 6b).

In addition, the sensitivity of the invention is also examined by probing the effective treatment time and concentration. As shown in FIG. 6d, the invention can detect the effect of rocaglamide in 25 nM, which is of similar sensitivity to other reported assays. More importantly, as fast as for only 15 mins, sufficient significant reduction of KRasG12V clustering on nanobars has been generated by the invention to reveal the drug effect, while 1 hr-incubation leads to a stabilized readout (FIG. 6c). In comparison, other established methods require much longer time to reach the final results, e.g., GTP-Ras pull down assay (12.5 nM to 200 nM, 24 hrs), FLIM-FRET (25 nM to 50 nM, 24 hrs) and EM (50 nM to 200 nM, 24 hrs). Therefore, the invention greatly shortens the assay time for drug screening without compensating the detection limit.

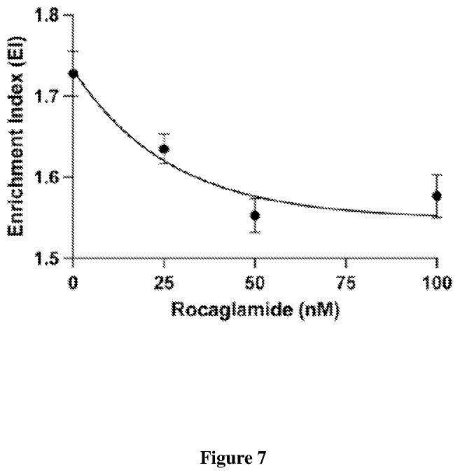

Furthermore, the IC50 of the nanobar assay in examining the effect of rocaglamide on K-Ras clustering is derived to be 18.41 nM (FIG. 7). It is in the similar range with the previous NanoBit assay measuring K-Ras activation (Hela cells, 4 hrs, 24.8 nM) and MTT assay to evaluate the inhibition on the growth of cancer cells (HCT-116/ASPC-1/Calu-1 cells, 24 hrs, 9.47 nM/10.25 nM/11.94 nM). Thus, the accuracy of the nanobar assay in examining the drug effect can be demonstrated.

### Example 6

### Evaluation of Nanobar Dimensions for Ras Detection

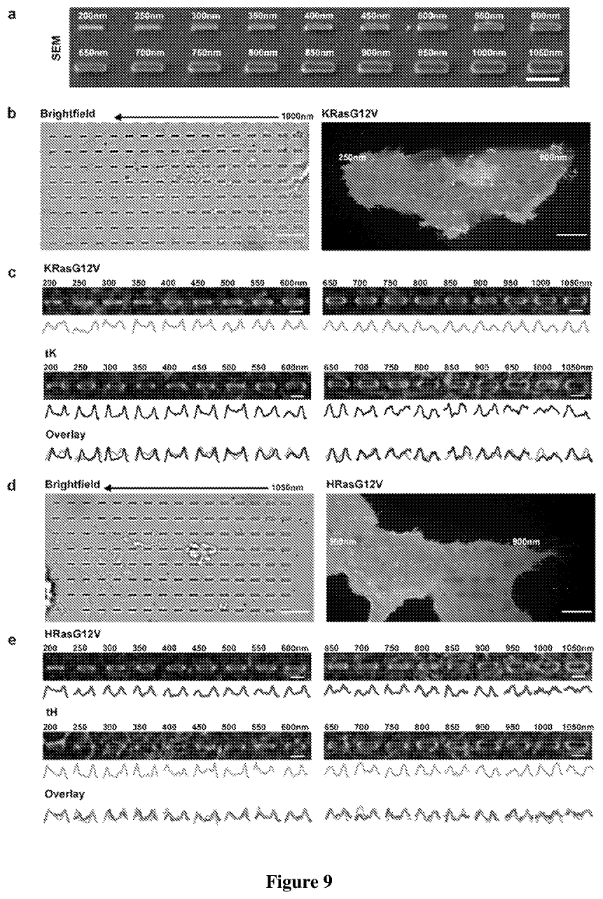

Besides the regular nanobars, the ability of nanostructures to guide Ras clustering has also been demonstrated on gradient nanobars through full-length KRasG12V, HRasG12V and the truncated Ras isoforms tK-Ras and tH-Ras. The gradient nanobars are designed with the diameter of curvature ranging from 200 to 1050 nm and a step size of 50 nm to create a gradient curvature sizes (FIG. 9a). The U2OS cells are cultured onto the gradient nanobar arrays, in which EGFP-tagged Ras variants are transiently expressed and their distributions are visualized (FIGS. 9b and d).

Then the Ras signals over multiple nanobars of each size are averaged. By plotting the intensity profiles of the averaged Ras distributions (FIGS. 9c and e), the effective curvature sizes that can guide Ras accumulation can be deduced. KRasG12V and HRasG12V started to accumulate at the nanobar diameter of 200 nm. KRasG12V continued to respond to the guidance of nanobars until the largest nanobar diameter tested, i.e. 1050 nm. In comparison, the curvature preference of HRasG12V to membrane curvature decays from the diameter of 1000 nm onwards. That is, the effective range of the nanobar guidance is 200 nm to 1050 nm diameter for KRasG12V and 200 nm to 950 nm diameter for HRasG12V.

Next, the comparison between the membrane anchors and the full-length Ras is made. For KRasG12V and tK, KRasG12V continues to respond to the guidance of nanobars until 1050 nm but tK decays its curvature preference since the diameter of 950 nm (FIG. 9c). That is, the effective range of the nanobar guidance is 200 nm to 1050 nm diameter for KRasG12V, whereas it is 200 nm to 900 nm diameter for tK. In addition, tK displays stronger signal accumulation to the nanobar ends for the nanobar diameters from 200 nm to 450 nm. Therefore, tK is more sensitive to the regulation of membrane curvature regulation within this curvature range.

For HRasG12V and tH, their sensitivities to different membrane curvatures are similar as their intensity profiles are mostly overlapping (FIG. 9e). The effective range that can guide their accumulation ranges from 200 nm to 1000 nm.

1. A method of detecting clustering of a Ras protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference indicates the isoform or mutation status of Ras.

2. The method of claim 1, wherein the Ras protein is an endogenous or heterologous Ras protein.

3. The method of claim 1, wherein the method comprises labelling the Ras protein with a fluorescence tag.

4. The method of claim 1, wherein the plurality of nanostructures comprises a combination of curved and flat surfaces.

5. The method of claim 1, wherein a curved surface has an arc width of less than about 1050 nm.

6. The method of claim 1, wherein the nanostructures are nanobars.

7. The method of claim 1, wherein a change in the degree of Ras clustering as compared to a reference indicates that the Ras protein has a G12V, G13D or Q61K mutation.

8. The method of claim 1, wherein a change in the degree of Ras clustering as compared to a reference indicates that the Ras protein is K-Ras, N-Ras or H-Ras isoform.

9. The method of claim 1, wherein the cell is treated with a drug molecule or candidate drug molecule.

10. The method of claim 9, wherein a change in the degree of Ras clustering as compared to a reference provides an indication of the specificity and/or sensitivity of an anti-Ras drug molecule or candidate drug molecule.

11. The method of claim 1, wherein a change in the degree of Ras clustering as compared to a reference predicts the biological activity of Ras in the cell.

12. A method of predicting the biological activity of Ras protein in a cell, the method comprising: a) culturing the cell on an array comprising a plurality of nanostructures; and b) visualizing the Ras protein using optical microscopy to detect clustering of the Ras protein around the plurality of nanostructures, wherein the Ras is labelled and wherein a change in the degree of Ras clustering as compared to a reference predicts the biological activity of Ras in the cell.

13. A method of identifying the isoform or mutation status of Ras in a cell, the method comprising: a) culturing the cell on an array comprising a plurality of nanostructures; and b) visualizing the Ras protein using optical microscopy to detect clustering of the Ras protein around the plurality of nanostructures, wherein the Ras protein is labelled, and wherein a change in the degree of Ras clustering as compared to a reference indicates the isoform or mutation status of Ras.

14. A method of identifying a subject suffering or is likely to be suffering from a cancer associated with a Ras mutation and/or isoform, the method comprising: a) culturing a cell from the subject on an array comprising a plurality of nanostructures; and b) visualizing the Ras protein using optical microscopy to detect clustering of the Ras protein around the plurality of nanostructures, wherein the Ras protein is labelled, and wherein a change in the degree of Ras clustering as compared to a reference identifies the subject as one suffering or is likely to be suffering from a cancer associated with a Ras mutation and/or isoform.

15. A method of screening for a drug compound, the method comprising: a) culturing a cell on an array comprising a plurality of nanostructures; b) contacting the cell with a candidate drug compound, c) visualizing Ras protein using optical microscopy to detect an increase or decrease in the degree of clustering of the Ras protein around the plurality of nanostructures as compared to a reference, wherein the Ras protein is labelled.

16. The method of claim 15, wherein the labelled protein is a membrane-associated protein.

17. The method of claim 15, wherein the drug compound is one that is capable of promoting or inhibiting clustering of the labelled protein.

18. The method of claim 15, wherein the cell is one that has been obtained from a subject.

19. A method of predicting the efficacy of an anti-Ras drug on a subject, the method comprising: a) culturing a cell that is obtained from the subject on an array comprising a plurality of nanostructures; b) contacting the cell with the anti-Ras drug, c) visualizing Ras protein using optical microscopy to detect an increase or decrease in the degree of clustering of the Ras protein around the plurality of nanostructures as compared to a reference, wherein the Ras protein is labelled, and wherein an increase or decrease in the degree of clustering of the Ras protein predicts the efficacy of the anti-Ras drug on the subject.

20. The method of claim 14, wherein the method comprises treating the subject identified as one suffering or is likely to be suffering from a cancer associated with a Ras mutation and/or isoform.
